(19) United States
(12) Patent Application Publication
(10) Pub. No.: US 2004/0152172 A1
(43) Pub. Date: Aug. 5, 2004

(54) Method for generation of a random RNAi library and its application in cell-based screens

(76) Inventor: Martin Geppert, Kalamazoo, MI (US)

Correspondence Address: PHARMACIA & UPJOHN, 301 HENRIETTA ST, 0228-32-LAW, KALAMAZOO, MI, 49007, US

(21) Appl. No.: 10/655,827

(22) Filed: Sep. 5, 2003

Related U.S. Application Data
(60) Provisional application No. 60/412,261, filed on Sep. 20, 2002.

(51) Int. Cl. C12P 19/34; C12N 15/85

(52) U.S. Cl. 435/91.2; 435/455

(57) ABSTRACT

The present invention provides methods and compositions relating to inhibitory RNA.

### CROSS REFERENCE TO RELATED APPLICATIONS

This application claims the benefit of the following provisional application: Application Serial No. 60/412,261, filed 20 Sep. 2002, under 35 U.S.C. 119(e)(1).

### FIELD OF THE INVENTION

The present invention provides methods and compositions relating to inhibitory RNA.

### BACKGROUND OF THE INVENTION

The complete genome sequences and large numbers of predicted gene sequences from many complex organisms are now available. Reverse genetic analyses of these organisms will now be necessary to understand the functions of all these genes and how they interact with each other. One of the major goals of every pharmaceutical company's R&D operation is to capitalize on these resources and to develop and implement genomics-based technologies that will accelerate target identification and validation for drug discovery purposes.

### LITERATURE CITED

1. Ambros, V Cell 107, 823 (2001)

2. Babinet, C & Cohen-Tannoudji, M An Acad Bras Cienc 73, 365 (2001)

3. Bargmann, CI Genome Biol 2 (2001)

4. Bass, BL Cell 101, 235 (2000)

5. Baulcornbe, DC Science 290, 108 (2000)

6. Bernstein et al. Nature 409, 363 (2001)

7. Brummelkamp et al. Science 296, 550 (2002)

8. Cogoni, C & Macino, G Curr Opin Genet Dev 10, 638 (2000)

9. Eggan et al. PNAS 98, 6209 (2001)

10. Eggan et al. Nat Biotechnol 20, 455 (2002)

11. Elbashir et al. Nature 411, 494 (2001)

12. Fire, A Trends Genet 15, 358 (1999)

13. Fire et al. Nature 391, 806 (1998)

14. Fraser et al. Nature 408, 325 (2000)

15. Fussenegger M Biotechnol Prog 17, 1 (2001)

16. Hannon, GJ Nature 418, 244 (2002)

17. Hrabe de Angelis et al. Nat Genet 25, 444 (2000)

18. Hrabe de Angelis M & Strivens M Brief Bioinform 2, 170 (2001)

19. Kasschau, KD & Carrington, JC Cell 95, 461 (1998)

20. Kawasaki et al. Nat Biotechnol 20, 376 (2002)

21. Ketting et al. Cell 99, 133 (1999)

22. Knight, SW & Bass, BL, Science 293, 2269 (2001)

23. Kolb, AF Cloning Stem Cells 4, 65 (2002)

24. Lagos-Quintana et al., Science 294, 853 (2001)

25. Lau et al., Science 294, 858 (2001)

26. Lee, RC & Ambros, V Science 294, 862 (2001)

27. Lee at al. Nat Biotechnol 19, 500 (2002)

28. Li, WX & Ding, SW Curr Opin Biotechnol 12, 150 (2001)

29. Lin, R & Avery, L Nature 402, 128 (1999)

30. Lipardi et al. Cell 107, 297 (2001)

31. Llave et al. PNAS 97, 13401 (2000)

32. Maeda et al. Curr Biol 11, 171 (2001)

33. Misra et al. BMC Biotechnol 1, 12 (2001)

34. Miyagishi, M & Taira, K Nat Biotechnol 19, 497 (2002)

35. Paddison et al., Genes Dev 16, 948 (2002)

36. Paul et al. Nat Biotechnol 19, 505 (2002)

37. Romano, N & Macino, G Mol Microbiol 6, 3343 (1992)

38. Ruvkun, G Science 294, 797 (2001)

39. Sharp, PA Genes Dev 15, 485 (2001)

40. Sijen et al. Cell 107, 465 (2001)

41. Sui et al. PNAS 99, 5515 (2002)

42. Tabara et al. Cell 99, 123 (1999)

43. Wianny, F and Zernicka-Goetz, M Nat Cell Biol 2, 70 (2000)

44. Zamore, PD Nat Struct Biol 8, 746 (2001)

### BRIEF DESCRIPTION OF THE FIGURES

FIG. 1 Structural features of random RNAi library of n-mers.

FIG. 2 Drawing depicting extending the population of random oligonucleotide RNAi progenitors via a polymerase extension reaction to produce a full hairpin random oligonucleotide RNAi progenitor.

FIG. 3 Drawing depicting denaturation of a full hairpin random oligonucleotide RNAi progenitor to generate a double stranded linear product and subsequent substantial removal of fixed primer sequences to generate an inhibitor sequence ready for cloning.

FIG. 4 Drawing depicting cloning of the inhibitor sequence ready for cloning.

### SUMMARY OF THE INVENTION

The invention provides a population of random oligonucleotide RNAi progenitors comprising a fixed primer sequence, a random oligonucleotide sequence and a fixed stem-loop structure. A preferred embodiment of the invention comprises a population of random oligonucleotide RNAi progenitors wherein the random oligonucleotide sequences are 15 to 50 bases in length. Especially preferred is a population of random oligonucleotide RNAi progenitors wherein the random oligonucleotide sequences are 20 to 30 bases in length. Even more preferred is a population of random oligonucleotide RNAi progenitors wherein the random oligonucleotide sequences are 21 to 23 bases in length.

The invention further provides a population of full hairpin random oligonucleotide RNAi progenitors comprising a double stranded fixed primer sequence, a double stranded random oligonucleotide sequence; and a fixed stem-loop structure. A preferred embodiment of the invention comprises a population of full hairpin random oligonucleotide RNAi progenitors wherein the random oligonucleotide sequences are 15 to 50 base pairs. Especially preferred is a population of full hairpin random oligonucleotide RNAi progenitors wherein the random oligonucleotide sequences are 20 to 30 base pairs in length. Even more preferred is population of full hairpin random oligonucleotide RNAi progenitors wherein the random oligonucleotide sequences are 21 to 23 base pairs in length.

The invention further provides a population of denatured full hairpin random oligonucleotide RNAi progenitors comprising a denatured fixed primer sequence, a denatured random oligonucleotide sequence and a denatured stem-loop structure. A preferred embodiment of the invention comprises a population of denatured full hairpin random oligonucleotide RNAi progenitors wherein the denatured random oligonucleotide sequences are 15 to 50 bases in length. Especially preferred is a population of denatured full hairpin random oligonucleotide RNAi progenitors wherein the denatured random oligonucleotide sequences are 20 to 30 bases in length. Even more preferred is population of denatured full hairpin random oligonucleotide RNAi progenitors wherein the denatured random oligonucleotide sequences are 21 to 23 bases in length.

The invention further provides a population of inhibitor sequences ready for cloning comprising a double stranded random oligonucleotide sequence; and a double stranded fixed stem-loop structure. A preferred embodiment of the invention comprises a population of inhibitor sequences ready for cloning wherein the denatured random oligonucleotide sequences are 15 to 50 bases in length. Especially preferred is a population of inhibitor sequences ready for cloning wherein the denatured random oligonucleotide sequences are 20 to 30 bases in length. Even more preferred is a population of inhibitor sequences ready for cloning wherein the denatured random oligonucleotide sequences are 21 to 23 bases in length. The invention further comprises a population of vectors comprising a population of inhibitor sequences ready for cloning.

The invention further provides a method to generate a population of inhibitor sequences ready for cloning comprising extending the population of random oligonucleotide RNAi progenitors via a polymerase extension reaction to produce a full hairpin random oligonucleotide RNAi progenitor, denaturing said full hairpin random oligonucleotide RNAi progenitor to produce a denatured full hairpin random oligonucleotide RNAi progenitor, extending said denatured full hairpin random oligonucleotide RNAi progenitor via a polymerase extension reaction to create a double stranded linear product and removing primer sequences from said double stranded product.

### DETAILED DESCRIPTION OF THE INVENTION

Conditional and targeted genetic knockout technologies are powerful reverse genetic tools but are expensive and relatively slow to accomplish in the preferred mammalian model organism, the house mouse (Babinet and Cohen-Tannoudji (2001)). Several shortcuts to gene inactivation are being tried. These include new technologies for generating targeted disruptions including the use of new recombinases (Kolb (2002)), tetraploid embryo aggregations (Misra et al. (2001); Eggan et al. (2002); Eggan et al. (2001)) and inducible expression systems (Fussenegger (2001)). Likewise, forward genetic tools like genome-wide mutagenesis using ENU or trap vectors (Hrabe de Angelis et al. (2000); Hrabe de Angelis and Strivens (2001) or mutagenesis of ES cells using EMS though powerful are labor intensive, relatively slow and expensive.

The advent of RNA interference (RNAi) technology has been hailed as a major breakthrough for studying gene function and to identify and validate “drugable” targets, not only in these model organisms, but also in organisms previously considered not being amenable to genetic analysis.

RNAi is a powerful tool in the arsenal of reverse genetics technology to ablate or significantly reduce gene function in vertebrate cells or whole organisms. It is a highly conserved mechanism of post-transcriptional gene silencing in which double stranded (ds) RNA corresponding to a gene or gene coding region of interest is introduced into an organism, resulting in degradation of the corresponding mRNA (Fire (1999); Baulcombe (2000); Bass (2001); Sharp (2001); Hannon (2002)). Unlike antisense technology, in the nematode C. elegans the RNAi phenomenon persists for multiple cell divisions (described below) before gene expression is regained (Fire (1998). RNA interfernce is an ancient system that is found in both plant and animal kingdoms (Cogoni and macino (2000)), and has been proposed to be an evolutionarily conserved defense against viruses (Li and Ding (2001), several of which have double stranded RNA (ds RNA) genomes, as well as modulation of transposon activity (Kasschau and Carrington (1998); Llave et al. (2000); Tabara et al. (1999); Ketting et al. (1999)) and regulation of gene expression (Lin and Avery (1999); Ruvkun (2001)). The phenomenon is described as RNAi in animals, post-transcriptional gene silencing (PTGS) in transgenic plants, VIGS in virus-infected plants (Zamore (2001)) and ‘quelling’ in fungi (Romano and Macino (1992)).

While PTGS phenomenon has been known for more than a decade, the mechanism of RNAi is only now beginning to be understood. The current model holds that after introduction into susceptible cells, dsRNA is recognized and cleaved into fragments of 21-25 nt by an RNase-III-like endonuclease called Dicer in an ATP dependent reaction (Bernstein et al (2001); Knight and Bass (2001)). The complex of proteins formed on the duplex siRNA is denoted ‘siRNP” to distinguish it from the fully active “RNA-induced silencing complex” (RISC) capable of cleaving its RNA target (Bernstein et al. (2001). These siRNAs anneal with target mRNAs and can cause destruction in two ways. First, the complexes are recognized by RNAseIII like enzymes, helicases etc. and the mRNA is cleaved at a point that corresponds roughly to the center of the siRNA. Alternatively, in C elegans, after the siRNA anneals with the mRNA, it is elongated by an RNA-dependent RNA polymerase. The endonuclease Dicer then generates a new round of siRNAs that, in a self-perpetuating process, go on to target further mRNAs. Therefore, in C. elegans, the phenomenon of RNAi is aptly described as degradative PCR because this RNA dependent RNA polymerase chain reaction, primed by siRNA, amplifies the interference caused by a small amount of ‘trigger’ ds RNA (Lipardi et al. (2001); Sijen et al. (2001)).

The first description of RNAi in a mammalian system was published by Wianny and Zemicka-Goetz in early 2000. In the meantime, several publications have confirmed this result and expanded further on the mechanistic nature of interference. In mammalian cells, dsRNA is processed into siRNA, but RNAi with dsRNA has not been particularly successful in most cell types because of nonspecific responses elicited by dsRNA molecules longer than 30 nt, most probably due to activation of the PKR pathway. More recently, there have been reports that transfection of synthetic 21-nt siRNA duplexes into mammalian cells effectively inhibits endogenous target gene expression in a highly sequence specific manner (Elbashir et al. (2001); Paddison et al. (2002). This was followed by a large number of reports that demonstrated efficacy in a variety of cell types of mammalian expression vector-mediated synthesis of siRNAs for knockdown of target genes (Brummelkamp et al. (2002); Paddison et al. (2002); Sui et al. (2002); Paul et al. (2002); Miyagishi & Taira (2002); Lee et al. (2002)). Also, the recent discovery of a large number of microRNA (miRNA) genes raises the prospect that the cellular machinery for siRNA inhibition in mammalian cells may be linked to normal processes of gene regulation (Lagos-Quintana et al. (2001); Lau et al. (2001); Lee and Ambros (2001); Ambros (2001)).

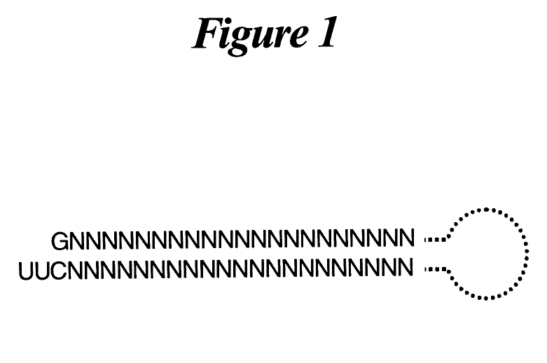

While many academic and industry laboratories are using RNAi as a reverse genetics tool to elucidate the function of specific genes or even the entire complement of a genome (for C. elegans: Fraser et al. (2000); Bargmann (2001); Maeda et al. (2001)), there is no report or review that proposes or describes a comprehensive protocol for a forward genetics application of RNAi. For a recent review see Hannon 2002. Such an application requires the generation of a library of random siRNA molecules in a suitable expression system. Random siRNAs typically would consist of double-stranded RNA sequence of random composition (N's) whereby the two strands are connected via a loop region of a variable number of base pairs (represented as a dotted line loop). It is thought that the enzyme “Dicer” further processes this molecule by cleaving off the loop region (FIG. 1).

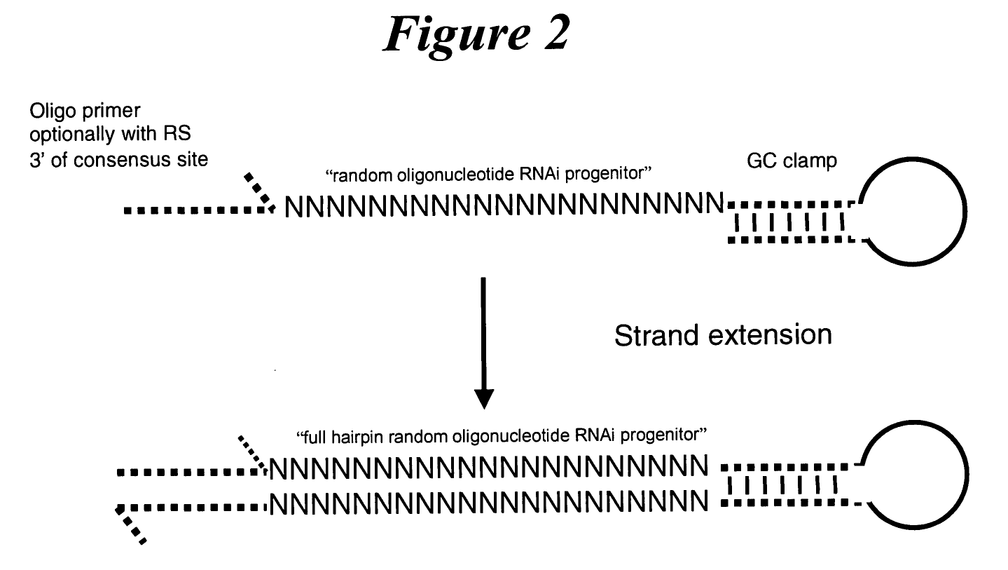

Here, a method is described that will allow rapid cloning of a random siRNA library based on a PCR based approach (FIG. 2 to 4).

A “random oligonucleotide RNAi progenitor” is synthesized incorporating the following features: a) a fixed primer sequence of sufficient length and suitable sequence composition to act as a primer under conditions suitable for the polymerase extension reaction described below. Optionally the fixed primer sequence may incorporate a restriction site within or at the 3′ of the sequence (dashed line), a stretch of random oligonucleotide sequence, of between 15 and 50 bases in length, preferentially 21-23 bases in length (N's) and a fixed stem-loop structure (solid black). The stem may be rich in GC content (“GC clamp”). The 3′ end of the stem-loop will serve as starting point for the next step. By “random oligonucleotide sequence” it is meant that the pool of nucleotide sequences of a particular length does not significantly deviate from a pool of nucleotide sequences selected in a random manner (i.e., blind, unbiased selection) from a collection of all possible sequences of that length. However, it is recognized that the sequences of random oligonucleotides may not be entirely random in the mathematic sense. Chemically synthesized random oligonucleotides will be random to the extent that physical and chemical efficiencies of the synthetic procedure will allow.

“Strand extension” refers to the process of elongation of a primer on a nucleic acid template. Using appropriate buffers, pH, salts and nucleoside triphosphates, a template dependent polymerase such as a DNA polymerase incorporates a nucleotide complementary to the template strand on the 3′ end of a primer which is annealed to a template. The polyrnerase will thus synthesize a faithful complementary copy of the template. Suitable polymerases for this purpose include but are not limted to E. coli DNA polymerase I, Klenow fragment of E. coli polymerase I, T4 DNA polymerase, other available DNA polymerases, polymerase muteins reverse transcriptase, and other enzymes, including heat-stable enzymes (i.e. those enzymes which perform primer extension after being subjected to temperatures sufficiently elevated to cause denaturation, for example Taq polymerase). Suitable enzymes will facilitate combination of the nucleotides in the proper manner to form the primer extension products which are complementary to each mutant nucleotide strand.

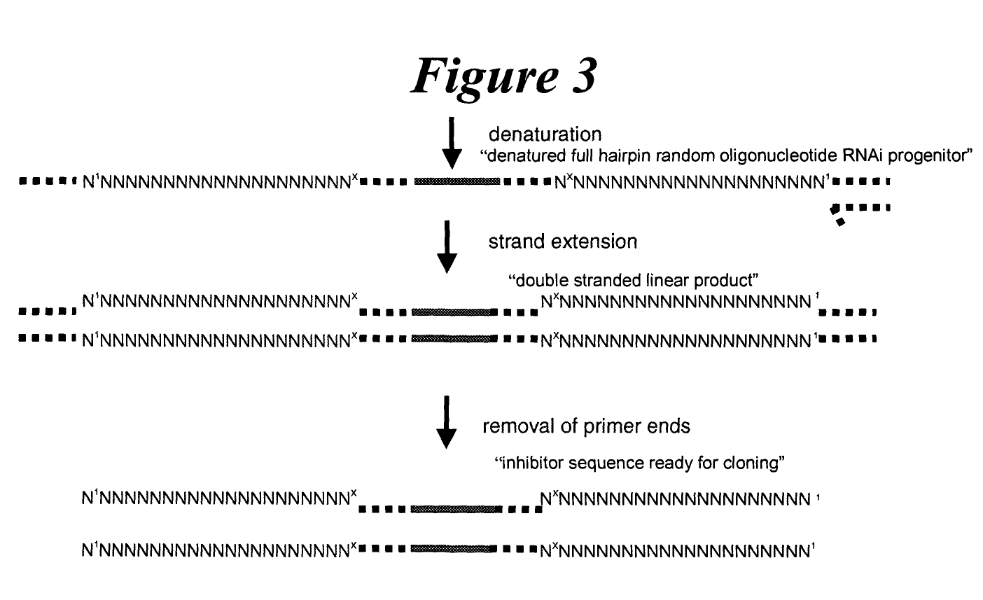

The “full hairpin random oligonucleotide RNAi progenitor” is subject to the following treatments: a) Denaturation, often times thermal denaturation, (FIG. 3, top panel) to break up all base pairing. b) polymerase extension after annealing of a oligonucleotide primer and “strand extension” by (FIG. 3, middle panel) to produce a “double stranded linear product”. Removal of primer sequences is accomplished by digestion with restriction enzyme(s) or nucleases (FIG. 3, bottom panel) to produce a “sequence ready for cloning”.

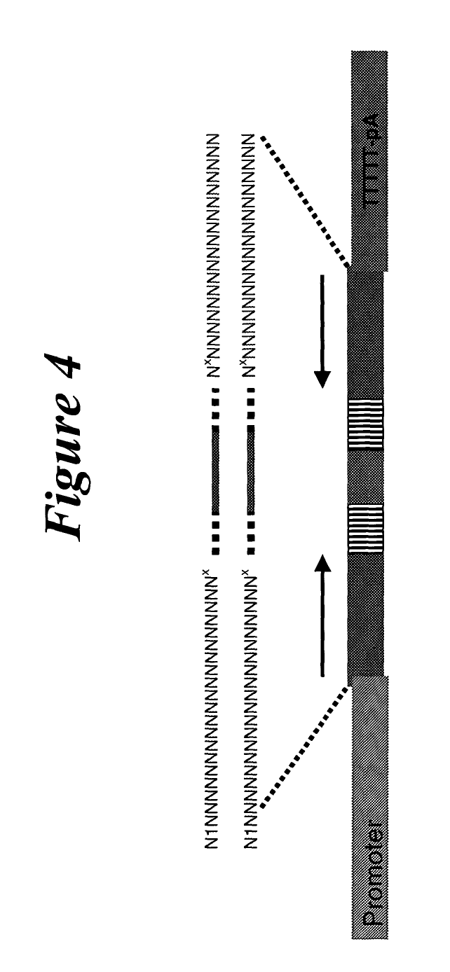

The product of the preceding procedure (“inhibitor sequence ready for cloning”) is cloned into a vector that allows constitutive or inducible expression of the siRNA-encoding sequences. (FIG. 4) after introduction into a suitable cell type. Methods for introducing DNA into a cell that are well known and routinely practiced in the art include transformation, viral infection, transfection, electroporation, nuclear injection, or fusion with carriers such as liposomes, micelles, ghost cells, and protoplasts. Expression systems of the invention include bacterial, yeast, fungal, plant, insect, invertebrate, vertebrate, and mammalian cells systems.

RNAi chips or other solid supporting material can be fabricated—arrays of siRNA on which cultured cells of many types can be grown and scored for the effects of suppressing expression of every gene in the genome, one-by-one. With the random RNAi library described above, RNAi technology can be taken one step further and incorporated into forward genetic screens for cellular loss of function/hypomorphic phenotypes that are of particular interest in biomedical research.

It is envisioned that any number of cellular phenotypes could be screened for after delivery of the random RNAi library to the appropriate cell types. Some phenotypes specifically envisioned include but are not limited to resistance to: induction of apoptosis, induction of a transformed phenotype, differentiation, chemotherapeutics oxidative stress, ER stress and angiogenesis (embryoid bodies) Platforms like ArrayScan or KineticScan setups may also be used for high throughput screening for phenotypes other than survival of a certain challenge.

Once cell clones displaying the desired phenotype are identified (e.g. resistance to apoptosis induced by TNFα or serum withdrawal), the siRNA responsible for the phenotype can be PCR amplified and sequenced. A BLAST search of the derived sequence against the relevant genome sequence should identify the target mRNA whose knockdown resulted in the cellular phenotype.

Example 1 below outlines a phenotypic screen, with embryonic stem (ES) cells characterizing “survivors” after TNFα challenge (Kawasaki et al. (2002).

### EXAMPLE 1

To determine the identity of genes involved in TNFα induced cell death. The following steps are envisioned:

One would first determine a preferred vector for ES cell transfection (or any other primary or immortalised cell line of interest). One would then electroporate/infect cells of choice and double-select (antibiotic resistance marker & survivor phenotype of interest [e.g. resistance to TNFalpha induced apoptosis]). The siRNA sequence from genomes of resistant clones is then amplified by PCR. Optionally, multiple rounds of screening could be performed. A BLAST search of the appropriate genome is performed to identify the target mRNA. Optionally one might attempt to rescue the phenotype with an expression plasmid containing target cDNA or BAC (e.g. in this example to re-instate susceptibility to TNFalpha). Optionally once the human orthologue identified the function of the identified transcript can then be further investigated e.g. to determine the potential role of the transcript in human disease (cancer etc.)

It will be clear that the invention may be practiced otherwise than as particularly described in the foregoing description and examples.

Numerous modifications and variations of the present invention are possible in light of the above teachings and, therefore, are within the scope of the invention.

The entire disclosure of all publications cited herein are hereby incorporated by reference to the extent not inconsistent with the disclosure herein.

What is claimed is:

1. A method to generate a population of inhibitor sequences ready for cloning comprising:
a.) extending a population of random oligonucleotide RNAi progenitors comprising
a fixed primer sequence;
a random oligonucleotide sequence; and
a fixed stem-loop structure;
via a polymerase extension reaction to produce a full hairpin random oligonucleotide RNAi progenitor;
b. denaturing said full hairpin random oligonucleotide RNAi progenitor to produce a denatured full hairpin random oligonucleotide RNAi progenitor;
c. extending said denatured full hairpin random oligonucleotide RNAi progenitor via a polymerase extension reaction to create a double stranded linear product and
d. removing primer sequences from said double stranded product.

2. The method of claim 1 further comprising inserting said product into an expression vector.

3. The method of claim 2 further comprising introducing said expression vector into a cell.

4. The method of claim 3 wherein said cell is assessed for a phenotype.

5. The method of claim 4 wherein said phenotype is a loss of function phenotype.

6. The method of claim 4 wherein the said phenotype is a partial loss of function phenotype.

7. The method of claim 4 wherein the said phenotype is due to the loss of function of a receptor gene.

8. The method of claim 4 wherein the said phenotype is due to the partial loss of function of a receptor gene.

9. The method of claim 1 wherein the population of sequences ready for cloning comprises a denatured random oligonucleotide sequence of 15 to 50 bases in length.

10. The method of claim 1 wherein the population of sequences ready for cloning comprises a denatured random oligonucleotide sequence of 20 to 30 bases in length.

11. The method of claim 1 wherein the population of sequences ready for cloning comprises a denatured random oligonucleotide sequence of 21 to 23 bases in length.
